(19) United States
(12) Patent Application Publication
(10) Pub. No.: US 2013/0055830 A1
(43) Pub. Date: Mar. 7, 2013

(54) APPARATUS AND METHOD FOR MEASUREMENT OF HAND JOINT MOVEMENT

(71) Applicants: Cheryl Metcalf, Eastleigh (GB); Scott Notley, Pulborough (GB)

(72) Inventors: Cheryl Metcalf, Eastleigh (GB); Scott Notley, Pulborough (GB)

(73) Assignee: UNIVERSITY OF SOUTHAMPTON, Southampton (GB)

(21) Appl. No.: 13/583,455

(22) PCT Filed: Mar. 8, 2011
(86) PCT No.: PCT/GB2011/050457

§ 371 Date: Nov. 16, 2012

(30) Foreign Application Priority Data
Mar. 9, 2010 (GB) 1003883.4

(51) Int. Cl. A61B 5/00

(52) U.S. Cl. 73/865.4

(57) ABSTRACT

Signal processing apparatus (1) for measuring hand joint movement comprising a plurality of markers (5) located at particular positions on a hand (20) and further comprising monitoring apparatus (10) to monitor movement of the markers to obtain dynamic positional information of the markers, and the apparatus further comprising a processor (12) to process the positional information to determine hand joint movement, wherein the processor configured to use the positional information of the markers to determine planes associated with respective groups of markers, wherein the processor configured to determine a first plane and a second plane, said planes adjacent to a hand joint, the first plane is substantially determined by a respective group of markers, and the processor configured to determine the second plane by reference to the first plane and the processor further configured to determine a change in angle between the two planes as a result of hand joint movement.

### TECHNICAL FIELD

The present invention relates generally to an apparatus and method for the measurement of hand joint movement.

### BACKGROUND

Various systems are known for measuring the complex movements of the hand. Known systems comprise the use of markers in motion analysis techniques in which the markers are positioned at particular locations on a subject's hand. As the subject moves his hand, for example to perform various prehension activities, such as pick and place activities, the movement of the markers (therefore also movement of the hand) is recorded. The movement of the markers is recorded by a suitable image recording arrangement, such as a plurality of cameras. However, known systems can provide varying degrees of reliability and can be cumbersome to use.

Known kinematic measurement techniques comprise either over-simplified methods that concentrate on specific joint angles (such as those that only calculate wrist joint angles or the joint angles of the index finger), or they can be extremely complex interpretations of a series of joints in the kinematic chain. Such known methods, although useful, are limited in that associated marker placement protocols can be very complex and can often include static splints or rod-based marker systems, which restrict or interfere with the natural movement of the joints.

We seek to provide an improved apparatus and method for measuring hand joint movement.

### SUMMARY

According to a first aspect of the invention there is signal processing apparatus for measuring hand joint movement comprising a plurality of markers located at particular positions on a hand and further comprising monitoring apparatus to monitor movement of the markers to obtain dynamic positional information of the markers, and the apparatus further comprising a processor to process the positional information to determine hand joint movement, wherein the processor configured to use the positional information of the markers to determine planes associated with respective groups of markers, wherein the processor configured to determine a first plane and a second plane, said planes adjacent to a hand joint, the first plane is substantially determined by a respective group of markers, and the processor configured to determine the second plane by reference to the first plane and the processor further configured to determine a change in angle between the two planes as a result of hand joint movement.

The processor is preferably configured to determine a respective vector for each plane, which vector projects from the respective plane.

The processor may be configured to determine first component vectors within the first plane, the processor further configured to determine to use the first component vectors to determine the vector projecting from the first plane.

The processor may be configured to determine second component vectors within the second plane, and wherein the processor further configured to determine the second component vectors in relation to the first component vectors, and the processor further configured to use the second component vectors to determine a vector projecting from the second plane.

The processor may be configured to substantially align each second vector component with a respective corresponding first component vector.

The processor is preferably configured to determine a third plane which includes, and is substantially defined by, a second group of markers, and the processor configured to determine the second component vectors by modifying the component vectors of the third plane in relation to the respective corresponding component vectors of the first plane.

The processor is preferably configured to determine the first plane as being the plane which is closer to the forearm of the subject.

The markers are preferably located at at least some of the following locations:
distal head of the ulnar
distal head of the radial styloid process
dorsal aspect of the ulnar
dorsal aspect of the radius
Proximal head of the first metacarpal at the carpometacarpal joint
Proximal head of the second metacarpal at the carpometacarpal joint
Proximal head of the fifth metacarpal at the carpometacarpal joint
Distal head of the first metacarpal
Distal head of the second metacarpal
Distal head of the third metacarpal
Distal head of the forth metacarpal
Distal head of the fifth metacarpal
Distal head of the proximal phalanx of the thumb
Distal head of the distal phalanx of the thumb
Distal head of the proximal phalanx of the second finger
Distal head of the medial phalanx of the second finger
Distal head of the distal phalanx of the second finger
Distal head of the proximal phalanx of the third finger
Distal head of the medial phalanx of the third finger
Distal head of the distal phalanx of the third finger
Distal head of the proximal phalanx of the fourth finger
Distal head of the medial phalanx of the fourth finger
Distal head of the distal phalanx of the fourth finger
Distal head of the proximal phalanx of the fifth finger,
Distal head of the medial phalanx of the fifth finger, and
Distal head of the distal phalanx of the fifth finger

Wherein, the second finger to the fifth finger are located progressively further away from the thumb.

According to a second aspect of the invention there is provided a method of measuring hand joint movement comprising receiving positional information signals from markers located at positions on a subject's hand, using the positional information to determine first and second planes, each plane associated with respective groups of markers, the groups of markers adjacent to a hand joint, determining the first plane substantially with reference to a plane defined by a first group of markers and determining the second plane with reference to the first plane, and determining the change in angle between the planes which occurs as a result of hand joint movement.

According to a third aspect of the invention there is provided machine readable instructions for a processor of a signal processing apparatus for measuring hand joint movement, the instructions being such that, when executed by the processor the instructions cause the processor to use the positional information signals from markers located on a subject's hand to determine first and second planes, each plane associated with respective groups of markers, the groups of markers adjacent to a hand joint, the instructions also so as to cause the processor to determine the second plane with reference to the first plane, and the instructions further so as to calculate a change in angle between the planes which occurs as a result of the hand joint movement. The machine readable instructions may conveniently be stored on any suitable data carrier, or may be embodied as a software product.

### BRIEF DESCRIPTION OF THE DRAWINGS

Various embodiments of the invention will now be described, by way of example only, with reference to the following drawings in which:

FIG. 1 is a view of apparatus for measuring hand joint movement,

FIG. 2 is a view of a hand provided with a plurality of markers,

FIG. 3 is a table of marker positions,

FIG. 4 shows planes and vectors generated to calculate joint movement,

FIG. 5 shows a schematic representation of two planes, and

FIG. 6 shows a flow diagram.

### DETAILED DESCRIPTION

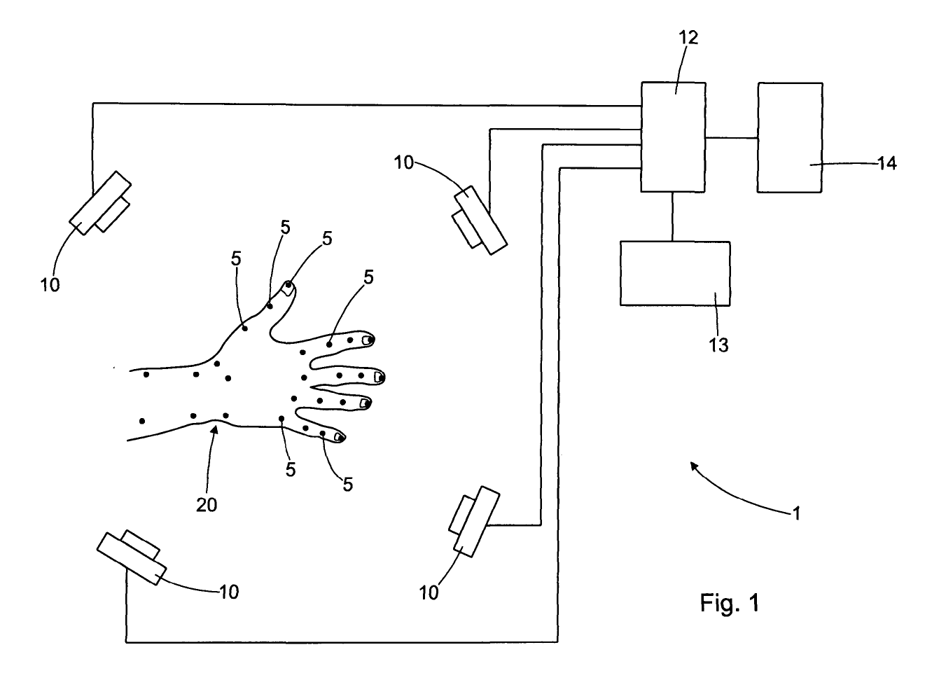

Reference is initially made to FIG. 1 which shows signal processing apparatus 1 for measuring hand joint movement. The apparatus 1 comprises monitoring apparatus comprising a plurality of cameras 10, a processor 12, a data input device 13 for the processor 12 and a data output device 14 for the processor 12. Associated with the processor 12 there is provided a memory to store instructions to configure the processor to perform the required processing of signals received from the cameras. The apparatus 1 further comprises a plurality of markers 5 which are attached to the skin of a subject's hand 20. As will be described in detail below, as the subject moves his hand, the cameras monitor movement of the markers. This dynamic positional information of the markers thus obtained is then processed by the processor 12 and reliably accurate data on movement of a particular joint is obtained.

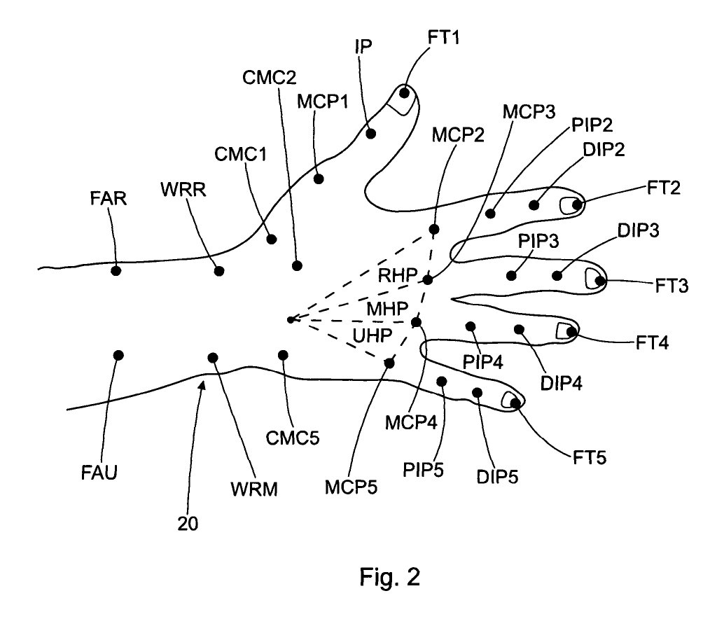

The markers 5 are hemispherical passive reflective markers. The markers are placed at the following locations on the subject's hand, as shown in FIG. 2:
Distal head of the ulnar (WRU)
Distal head of the radial styloid process (WRR)
Dorsal aspect of the ulnar (FAU)
Dorsal aspect of the radius (FAR)
proximal head (CMC1) of the first metacarpal at the
carpometacarpal (CMC) joint,
proximal head (CMC2) of the second metacarpal at the CMC joint,
proximal head (CMC5) of the fifth metacarpal at the CMC joint,
distal head (MCP1) of the first metacarpal,
distal head (MCP2) of the second metacarpal,
distal head (MCP3) of the third metacarpal,
distal head (MCP4) of the forth metacarpal,
distal head (MCP5) of the fifth metacarpal,
distal head (IP) of the proximal phalanx of the thumb,
distal head (FT1) of the distal phalanx of the thumb,
distal head (PIP2) of the proximal interphalangeal of the second finger,
distal head (DIP2) of the medial phalanx of the second finger,
distal head (FT2) of the distal phalanx of the second finger,
distal head (PIP3) of the proximal phalanx of the third finger,
distal head (DIP3) of the medial phalanx of the third finger,
distal head (FT3) of the distal phalanx of the third finger,
distal head (PIP4) of the proximal phalanx of the fourth finger,
distal head (DIP4) of the medial phalanx of the fourth finger,
distal head (FT4) of the distal phalanx of the fourth finger.
distal head (PIP5) of the proximal phalanx of the fifth finger,
distal head (DIP5) of the medial phalanx of the fifth finger,
distal head (FT5) of the distal phalanx of the fifth finger

Wherein, in the reference convention used above the second finger to the fifth finger are located progressively further away from the thumb.

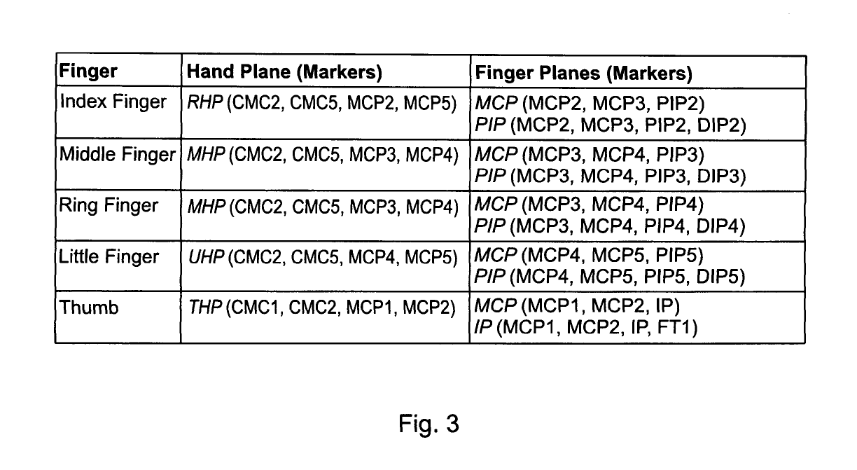

Three planes are defined in relation to the metacarpal arch, these planes being the radial hand plane (RHP), the middle hand plane (MHP) and the ulnar hand plane (UHP). These planes are shown in FIG. 2. The planes are constructed by the use of the MCP markers and a virtual marker, CMCVM, which is generated by the processor 12 at a position substantially halfway between the CMC2 and the CMC5 markers. FIG. 3 provides a summary of the planes and markers required for measuring the joint movement of different fingers. If it is required to measure movement of the wrist, using the above marker set, two planes can defined to achieve this. The markers FAU, FAR, WRR and WRU define a forearm plane and the markers CMC2, CMC5, MCP2 and MCP5 define a hand plane.

In total, twenty four degrees of freedom can be measured, these are flexion/extension and radial/ulnar deviation of the wrist, flexion/extension and abduction/adduction of the fingers at the metacarpophalangeal (MCP), flexion/extension at the proximal interphalangeal (PIP) and distal interphalangeal joints (DIP), flexion/extension of the transverse metacarpal arch, flexion/extension of the MCP and interphalangeal (IP) joint of the thumb, as well as abduction/adduction and rotation through to opposition of the thumb.

The monitoring apparatus comprises a motion analysis system such as a twelve-camera Vicon® T-series motion analysis system. The cameras of the system illuminate the hand with infrared radiation, and reflected radiation signals from the markers are received by the cameras. The positional information received by the cameras is sent to the processor 12 for analysis in order to calculate the movement of one or more hand joints. During an initial set up procedure, the processor 12 is configured to identify each of the markers. In this way the processor 12 is able to track the three-dimensional position (hence movement) of each marker in relation to a co-ordinate system.

Broadly, the processor 12 is configured to generate planes from particular groups of markers, which markers are located adjacent a hand joint of interest. The processor 12 is configured to then determine a respective (projected) normal vector associated with each plane. By analysing the movement of the two vectors the variation in angle subtended by the normal vectors is indicative of the movement of the joint under investigation. Creating the normal vector defines a local co-ordinates system (LCS) for that plane. It is the position of the LCS and the translation between adjacent LCSs that attributes to the accuracy of the measurement.

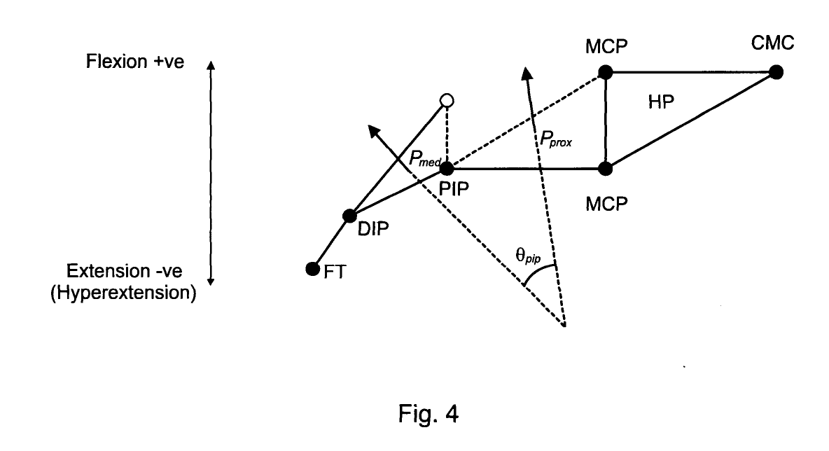

The above procedure of constructing planes and normal vectors from those planes is now further explained with reference to FIG. 4. FIG. 4 shows the groups of markers used to construct two planes, Pprox and Pmed, from which respective normal vectors are calculated, in order to calculate PIP joint flexion/extension. Specifically, to calculate PIP joint flexion/extension, a plane is created from the two vectors MCP2 to MCP3 and MCP2 to PIP2 (Pprox). A second plane is created from the two vectors MCP2 to MCP3 and PIP2 to DIP2 (Pmed). Since vectors have only magnitude and direction, and not position in space, the plane for the medial phalanx of the finger is also defined to move relative to the RHP during flexion and extension by anchoring the plane to the vector defined between the MCP joints (the second and third in this case). Therefore, any movement of the finger at the MCP joint will not have an effect on the PIP joint angle generated by this calculation method. The unit vectors normal to both planes are defined using equation (1) below and the PIP joint angle is calculated between the two normal vectors defined for the planes of the proximal and medial phalanges using equation (2) below.

$$\begin{matrix}{p}_{prox} = {v}_{mcp} \times {v}_{pip} & ( 1 ) \\ {p}_{med} = {v}_{mcp} \times {v}_{dip} \\ {θ}_{pip} = {cos}^{- 1} [ \frac{{np}_{prox} \cdot {np}_{med}}{\vert {np}_{prox} \vert \vert {np}_{med} \vert} ] & ( 2 )\end{matrix}$$

Where np_{ab} is the unit vector normal to the plane ab.

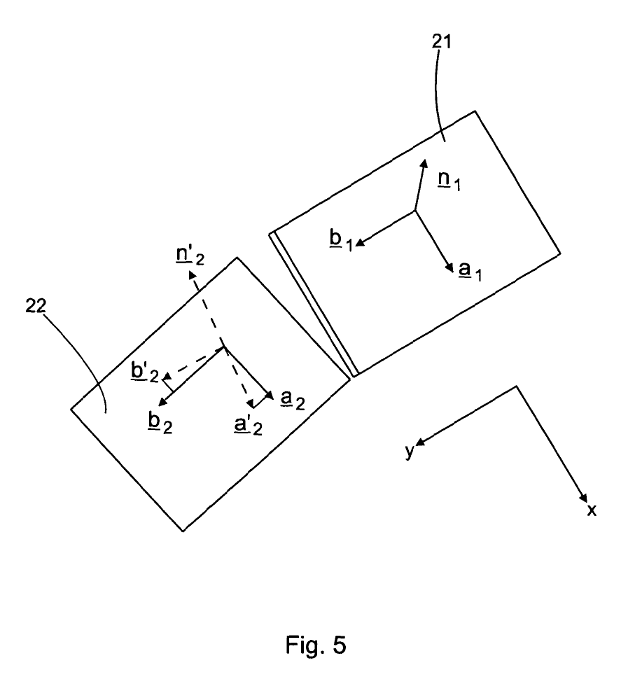

More specifically in relation to the processing steps above, we have appreciated that significantly more accurate results (of the movement of a hand joint) can be obtained from the positional information signals by adopting the processing steps, which are now further detailed. In overview, these steps essentially involve determining a normal vector associated with one plane which is determined by calculating ‘corrected’ component vectors (from which a ‘corrected’ normal vector associated with the plane is determined). For this, the planes adjacent to the joint of interest are referred to as the first plane and the second plane. The first plane 21 is that which is closest to the subject's forearm and the second plane is that which is further away from the subject's forearm. Reference is now made to FIG. 5. Within the first plane 21 two orthogonal component unit vectors a₁ and b₁ are defined. The vectors are co-directional with respective x and y axes, wherein the y-axis is the so-called long axis which extends generally longitudinally of the forearm. The second plane 22 includes two orthogonal unit component vectors a₂ and b₂. Whilst unit vectors a₁ and b₁ will be used to calculate a vector normal to the first plane 21, modified unit vectors, a′₂ and a′₂ based on the directions of unit vectors a₃ and b₂ will be calculated in order to determine a normal vector associated with the second plane 22. Component vector b′₂ is determined as a vector which is substantially co-directional with the corresponding respective vector of the first plane, namely b₁. In order to calculate b′₂ therefore the direction of b₂ is used. Similarly, a′₂ is determined by using the known direction of a₁.

The procedure of determining the modified unit vectors of the second plane is now further described. In general terms, the angular alignment of the two normal vectors P₁ and P₂ (defined by Pᵢ= x̅ᵢ̅×̅y̅ᵢ̅ iε{1,2} with component vectors x₁,y₁ and x₂,y₂ lie in respective planes) can be expressed with reference to any pair of orthogonal planes, each containing a selected one of the normal vectors. The angle of one of normal vectors to one of its planes is:

θⱼ=cos⁻¹( \overline{\hat{P}₂ⱼ·P₁}) jε{1,2}

Where \hat{P}_{2,j} is the projection of P₂ onto Aⱼ, given by:

\hat{P}_{2,j}=P₂∥Aⱼ jε{1,2}

To recover the direction of angular alignment, θⱼ is multiplied by

$$\{ \begin{matrix}1 & if \overline{{\hat{P}}_{2 , j} \vert \vert ( {P}_{1} \times {A}_{j} )} = {P}_{1} \times {A}_{j} \\ - 1 & otherwise\end{matrix}$$

By projecting one vector onto orthogonal planes containing the other normal vector, the other normal vector can be modified to be aligned with the first normal vector and so obtain a more accurate measurement of the angle of extension/flexion.

In order to calculate the movement of the joint, a normal vector n₁ is calculated by using an equation of the form of (1) using component vectors a₁ and b₁, and a normal vector n′₂ associated with the second plane is calculated using the same equation but with the (modified) vectors a′₁ and b′₂. The variation in angle subtended by the normal vectors during movement is then indicative of the movement of the joint. By using the first plane 21 as a reference co-ordinate system to construct modified unit vectors, the modified unit vectors are effectively ‘aligned’ with the component vectors of the first plane achieved by way of a transformation of the local co-ordinate system of vectors a₁ and b₁ applied to vectors a₂ and a₂, we are able to eliminate, or at least minimise, any error in measurement of the joint movement that would occur due to movement in another plane of movement (as opposed to the plane of movement in which we are primarily interested). As will be appreciated, joints of the hand are capable of movement in multiple axes and due to deformity or otherwise, movement of the hand may occur in more than one plane. The processing steps above of using modified unit vectors for the second plane enables such errors (occurring as a result of out-of-plane movement) to be reduced and so obtain significantly more accurate results.

It will be appreciated that the plane which includes the component vectors a′₂ and a′₂ is not co-planar with the plane which includes the (‘original’) component vectors a₂ and a₂.

In the case of the finger joint, when the plane of the medial phalanx passes the point of flexion through to extension (hyperextension in the case of the PIP joint) relative to the proximal phalanx, the resultant angle is negative (-ve) and is indicative of pathological movement. Thus, the method described here can provide evidence of PIP joint hyperextension due to swan-neck deformity during dynamic functional activities.

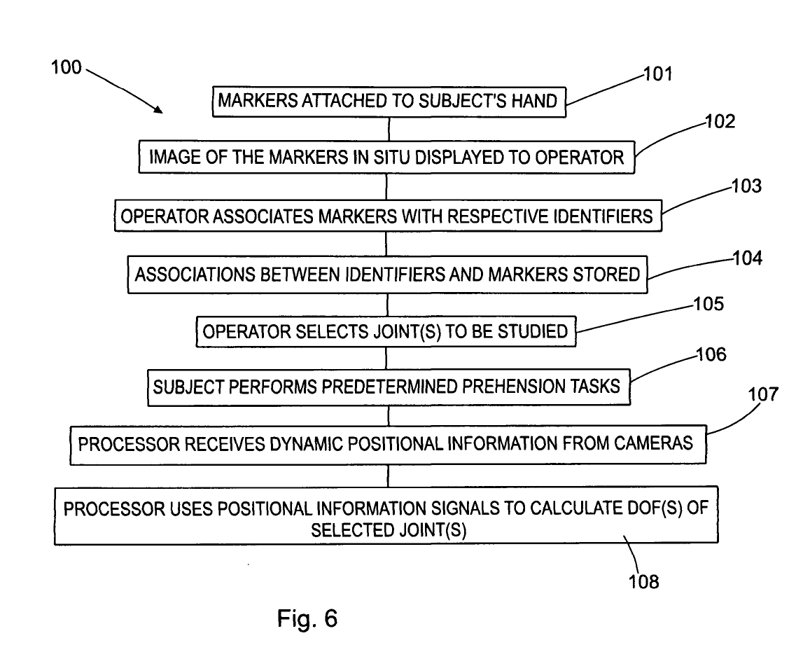

The apparatus 1 is used as follows, as described with reference to the flow diagram 100 shown in FIG. 6. At step 101, the operator attaches the markers 5 to the bony anatomical landmarks of a subject's hand in accordance with the placement protocol shown in FIG. 2. The cameras 10 receive reflected infra-red radiation from the markers, and images of the markers are shown to the operator on the output device 14, as stated at step 102. At step 103 the operator uses the input device 13 (which may be, for example a keyboard and/or mouse) to select the image of each marker and associate with each marker its respective identifier (for example, the identifier CMC1 in relation to the proximal head of the first metacarpal at the carpometacarpal (CMC) joint). This identifying information is received by the processor as an ASCII file. The memory associated with the processor 12 stores the relationships between the markers and their respective identifiers, as referred to at step 104. The instructions stored in the memory of the processor contain references to the marker identifiers and accordingly, the processor 12 is able to perform the necessary calculations by monitoring the three-dimension position of the relevant markers. The operator then uses the input device 13 to indicate to the processor 12 a selection of one or more joints, the degree(s) of freedom of which are to be studied, as shown at step 105. At step 106, the subject then performs a stipulated set of prehension tasks. As the prehension tasks are performed, the cameras 10 send positional information signals to the processor 12, as shown at step 107. As described above, the processor 12 the processes the received signals in accordance with the stored instructions, and in particular in relation to the joints selected by the operator at step 105. At processing step 108, the processor uses the received positional information to determine the degree(s)-of-freedom (DOF(s)) of the selected joint(s). The various processing steps performed by the processor may be summarised as follows:

(i) monitor change in position of relevant markers,
(ii) determine component vectors within a proximal plane,
(iii) use the component vectors to determine a unit vector which is normal to the proximal plane,
(iv) determine component vectors of second (distal) plane,
(v) modify the component vectors if the distal plane to align with corresponding respective component vectors of the first plane,
(vi) calculate the normal vector for the distal plane using the modified component vectors

Advantageously the use of the above marker set advantageously is intuitive, quick and simple to apply to a subject's hand. The marker set represents a relatively small marker set, and so this considerably eases the application of the markers to the subject's hand, and in particular from the subject's perspective. Furthermore, the use of projected angles (from generated planes) and a simple, anatomically defined marker set ensures a reliably accurate result. In the prior art, so-called Euler angles are used to calculate the angular range of movement of a joint in which three angles need to be calculated for each joint. This inevitably results in a greater processing complexity. In contrast, the use of projected angles described above considerably reduces processing complexity on the processor but ensures reliably accurate results. The apparatus 1 can be used to capture joint movement for a variety of applications, such as biomechanical investigations and animation production. In relation to biomedical investigations the improved accuracy will result in improved accuracy of analysis of the results output by the processor. Furthermore, in relation to animation production, improved accuracy will result in a more realistic rendering of hand movement.

1. Signal processing apparatus for measuring hand joint movement comprising a plurality of markers located at particular positions on a hand and further comprising monitoring apparatus to monitor movement of the markers to obtain dynamic positional information of the markers, and the apparatus further comprising a processor to process the positional information to determine hand joint movement, wherein the processor configured to use the positional information of the markers to determine planes associated with respective groups of markers, wherein the processor configured to determine a first plane and a second plane, said planes adjacent to a hand joint, the first plane is substantially determined by a respective group of markers, and the processor configured to determine the second plane by reference to the first plane and the processor further configured to determine a change in angle between the two planes as a result of hand joint movement.

2. Signal processing apparatus as claimed in claim 1 in which the processor configured to determine a respective vector for each plane, which vector projects from the respective plane.

3. Signal processing apparatus as claimed in claim 2 in which the processor configured to determine first component vectors within the first plane, the processor further configured to determine to use the first component vectors to determine the vector projecting from the first plane.

4. Signal processing apparatus as claimed in claim 3 in which the processor configured to determine second component vectors within the second plane, and wherein the processor further configured to determine the second component vectors in relation to the first component vectors, and the processor further configured to use the second component vectors to determine a vector projecting from the second plane.

5. Signal processing apparatus as claimed in claim 4 in which the processor configured to substantially align each second vector component with a respective corresponding first component vector.

6. Signal processing apparatus as claimed in claim 5 in which the processor configured to determine a third plane which includes, and is substantially defined by, a second group of markers, and the processor configured to determine the second component vectors by modifying the component vectors of the third plane in relation to the respective corresponding component vectors of the first plane.

7. Signal processing apparatus as claimed in claim 1 in which the processor configured to determine the first plane as being the plane which is closer to the forearm of the subject.

8. Signal processing apparatus as claimed in claim 1 in which a first normal vector associated with the first plane is projected onto orthogonal planes associated with the second normal vector and to thereby generate a modified second normal vector.

9. Signal processing apparatus as claimed in claim 1 in which the markers are located at at least some of the following locations:
distal head of the ulnar,
distal head of the radial styloid process,
dorsal aspect of the ulnar,
dorsal aspect of the radius,
proximal head of the first metacarpal at the carpometacarpal joint,
proximal head of the second metacarpal at the carpometacarpal joint,
proximal head of the fifth metacarpal at the carpometacarpal joint,
distal head of the first metacarpal,
distal head of the second metacarpal,
distal head of the third metacarpal,
distal head of the fourth metacarpal,
distal head of the fifth metacarpal,
distal head of the proximal phalanx of the thumb,
distal head of the distal phalanx of the thumb,
distal head of the proximal phalanx of the second finger,
distal head of the medial phalanx of the second finger,
distal head of the distal phalanx of the second finger,
distal head of the proximal phalanx of the third finger,
distal head of the medial phalanx of the third finger,
distal head of the distal phalanx of the third finger,
distal head of the proximal phalanx of the fourth finger,
distal head of the medial phalanx of the fourth finger,
distal head of the distal phalanx of the fourth finger,
distal head of the proximal phalanx of the fifth finger,
distal head of the medial phalanx of the fifth finger, and
distal head of the distal phalanx of the fifth finger,
wherein, the second finger to the fifth finger are located progressively further away from the thumb.

10. Signal processing apparatus as claimed in claim 1 in which the processor configured to determine the second plane using a transformation of a co-ordinate system local to the first plane.

11. A method of measuring hand joint movement comprising receiving positional information signals from markers located at positions on a subject's hand, using the positional information to determine first and second planes, each plane associated with respective groups of markers, the groups of markers adjacent to a hand joint, determining the first plane substantially with reference to a plane defined by a first group of markers and determining the second plane with reference to the first plane, and determining the change in angle between the planes which occurs as a result of hand joint movement.

12. Machine readable instructions for a processor of a signal processing apparatus for measuring hand joint movement, the instructions being such that, when executed by the processor the instructions cause the processor to use the positional information signals from markers located on a subject's hand to determine first and second planes, each plane associated with respective groups of markers, the groups of markers adjacent to a hand joint, the instructions also so as to cause the processor to determine the second plane with reference to the first plane, and the instructions further so as to calculate a change in angle between the planes which occurs as a result of the hand joint movement.
